(19) United States
(12) Patent Application Publication
(10) Pub. No.: US 2003/0221687 A1
(43) Pub. Date: Dec. 4, 2003

(54) Medication and compliance management system and method

(76) Inventor: William Kaigler, North Huntington, PA (US)

Correspondence Address: BLYNN L. SHIDELER, THE BLK LAW GROUP, 3500 BROKKTREE ROAD, SUITE 200, WEXFORD, PA, 15090, US

(21) Appl. No.: 10/422,060

(22) Filed: Apr. 23, 2003

Related U.S. Application Data
(60) Provisional application No. 60/378,839, filed on May 9, 2002.

(51) Int. Cl. A61M 11/00; A61M 16/00

(52) U.S. Cl. 128/200.14; 128/202.22; 128/204.23; 128/204.21

(57) ABSTRACT

A medical therapy compliance management system manages a patient's use of a therapy device, such as a nebulizer, in which treatments are to be provided at predetermined intervals. A compliance monitor coupled to the medical therapy device determines when a medical treatment is provided to the patient. A compliance controller receives data indicative of a medical treatment and can generate a reminder to the patient of future scheduled treatments. The compliance controller tracks the treatments provided and adjusts the reminders accordingly. The medical therapy compliance management system tracks certain parameters indicative of clinical or transactional events within the healthcare process. If any one of the measured or calculated parameters is outside a predetermined threshold the system notifies the patient and or caregiver. The system may also utilize interactive voice response technology to gather data from the patient.

### CROSS-REFERENCE TO RELATED APPLICATIONS

The present application claims the benefit of U.S. provisional patent application serial No. 60/378,839 filed May 9, 2002 entitled “Therapy Compliance Management System and Atomizer and/or Nebulizer Patient Interaction Appliance” which is incorporated herein by reference in its entirety.

### BACKGROUND OF THE INVENTION

1. Field of the Invention

The present invention relates to a medication and compliance management system and method. Specifically, the present invention relates to a respiratory medication monitoring and management and patient compliance management system and method for a nebulizer or the like.

2. Background Information

Medical research and associated advancements therein have resulted in significant improvements in patient quality of life and over all public health through the use of improved medication therapies. One of the largest problems facing such therapies is proper patient compliance to physician prescribed treatment regimens. This problem is increased with a larger number of patients taking therapies at home without the direct supervision of caregivers. Furthermore, it is costly and labor intensive to have caregivers, or others, repeatedly directly contact the patients (e.g. home visits, telephone calls or the like) for medication and compliance management. It should be readily apparent that a prescribed therapy is of no benefit to the patient if not taken, and of reduced benefit if not taken in the manner prescribed by the physician.

The result of poor compliance with a prescribed therapy by a patient is reduced patient quality of life and over all health. Further there are significant economic impacts of poor compliance, including increased expense associated with otherwise avoidable hospital admissions, increased hospital length of stays, increased doctor visits and inflated caregiver expenses associated with the patient's poorer overall health status.

Industry data, as well as respected clinical research, suggests that medication compliance, while influenced by a great number of potential contributing factors, tends to be reduced (less compliant behavior) in cases where the patient is either prescribed multiple medications and/or prescribed multiple treatments of the same medication. Medication compliance is obviously adversely affected when the patient does not obtain a timely refill of one medication. Further, patients experiencing impaired cognitive ability have an increased susceptibility to reduced compliance to medication therapy regimen.

While the medical community recognizes poor medication compliance as a significant problem, a general lack of quality and timely data limits the caregiver's ability to provide the necessary education and feedback to the patient to significantly affect the patient's compliance behavior at a reasonable cost. There is a significant need for managing patient medications by monitoring the patient to various degrees and managing the logistics with those patients (e.g. refills).

The medical community has developed a variety of patient compliance, patient reminder, patient controlling and patient monitoring systems. These systems specifically include oral (pill) medication compliance systems, medication reminder systems, technology to monitor and/or control patient treatment techniques for metered dose inhaler (MDI) systems and remote health monitoring technologies for disease management and clinical trial administering.

Regarding medication compliance systems, typical embodiments involve the management and monitoring of oral, or pill based, medications. Other inventions identified included the monitoring of diabetes injections and metered dose inhaler treatments. Regarding medication reminder systems there have been some systems designed to remind the patient of pre-arranged treatments on a timed or remote basis. Regarding remote health monitoring systems, there are a variety of systems intended to serve as a platform for disease management or the administering of clinical trials or studies. Finally regarding treatment techniques, existing systems focus on monitoring and providing feedback to the patient regarding the proper technique for a given therapy. A number of systems have been developed in this area for use with metered dose inhalers (MDI's).

Specifically, U.S. Pat. Nos. 6,270,455 and 6,168,563 discloses a remote monitoring networked system for communicating information to a patient and for remotely monitoring the patient. The system includes a server and a remote interface for entering in the server a set of queries to be answered by the patient.

U.S. Pat. No. 5,284,133 relates to a remote monitoring system and to specific treatment techniques disclosing an inhalation device with a control mechanism to assure patient compliance with a drug dosage regimen. An actuator, operating in conjunction with a timer, prevents the inhalation device from being actuated after the programmed dosage has been administered at the prescribed interval. A signaling device provides a patient reminder for inhaling his or her medicine at the prescribed time intervals. The history of actuation, non-actuation, and improper attempts at actuation can all be recorded and analyzed off-site.

U.S. Pat. No. 5,331,953 also discloses a remote monitoring system and specific treatment technique. A device used in connection with an inhaler is intended for measuring and recording the course of inhalation of a patient. A detector is provided in the inhaler in connection with a passage for the airflow of inhalation, whereby the detector detects the airflow of the inhalation through the inhaler as well as the availability of the medicine at the inhalation within said passage, so that a combination of these two detected values decides if and how the performed inhalation should be recorded in the electronic unit.

U.S. Pat. No. 4,572,403 discloses a dispensing system for timed dispensing of tablets, capsules and the like.

U.S. Pat. No. 6,102,855 discloses a medical monitoring device for managing administration of medical treatment regimens for treating a patient's medical conditions. The device stores medication schedule data, treatment data, patient query data, and patient response data. The device provides scheduled medication alarm signals that alert the user concerning prescribed medications due to be taken.

U.S. Pat. No. 5,809,997 discloses an electronic medication chronology device adapted for attachment to various shapes and sizes of conventional pressurized inhalant packages. The chronology device includes computing and recording equipment with audio and visual display mounted in a chronology housing.

U.S. Pat. No. 5,363,842 discloses a monitoring device that detects how much air is inhaled through an inhaler and at what time, including such derived measurements as how much volume is inspired within the bounds of a given flow range, as well as certain events such as the triggering of the release of aerosol.

U.S. Pat. No. 6,076,521 discloses a dose indicating device constructed for use with an inhalator. The inhalator includes a dosing unit defining an inhalation channel, and a maneuvering element. The system has an indicator constructed to detect movement of the maneuvering element and indicate to a user of the inhalator that a dose has been fed to the inhalation channel when the inhalator is activated for inhalation.

U.S. Pat. No. 4,984,158 discloses a visual and auditory biofeedback system that instructs patients in proper techniques of inhalation to administer aerosol drugs with Metered Dose Inhaler (MDI) devices. The system displays desired respiratory parameters along with real time performance. Performance records may be saved for clinical or research needs.

U.S. Pat. No. 5,842,468 discloses an apparatus for administering a fluid medicament to a patient in a gas for inhalation and for calculating the dosage administered to the patient. The apparatus includes a detector sensing the rate of flow of gas inhaled by a patient from the holding chamber and calculates the amount of medicament received by a patient.

U.S. Pat. No. 5,404,871 discloses an apparatus for delivering an amount of aerosolized medicine for inspiration by a patient in response to the occurrence of appropriate delivery point or points in the patient's detected breath flow. Changes in the patient's breath flow patterns during the course of an aerosolized medication inspiration therapy program may be detected and used to adjust the controlled amount of medication to be delivered in a given administration and/or to inform the patient of the patient's condition or change in condition. The device also may contain a library of administration protocols or operating parameters for different medications.

U.S. Pat. No. 5,019,974 discloses a system for efficient medical control of a medical condition such as diabetes that comprises a recorder, an interface and a master computer. The master computer develops a program of therapy which is downloaded into the recorder which then reminds the patient of any therapy due and records that the therapy has been affected. The record from the recorder is subsequently fed back to the master computer to improve or alter the therapy program.

U.S. Pat. No. 5,505,195 discloses a dry powder inhalant device adapted for mounting on a conventional medication dry powder dispenser. The device is designed for monitoring prescribed dosages of dry powder medication received. The device includes an electronic housing mounted on the dispenser for computing and recording when a proper amount of dry powder is released inside the dispenser, when a proper amount of air flow is inhaled through the dispenser for mixing with the dry powder, and when each dispenser or dry powder container is removed and replaced on the electronic housing.

U.S. Pat. No. 4,817,822 discloses a device for indicating the number of doses dispensed from an aerosol container.

U.S. Pat. No. 5,042,685 discloses a medication dispenser that includes a receptacle in which openings are blocked or unblocked by logic built into or remotely connected to the dispenser, wherein the logic determines whether the pills in the compartment should be dispensed.

U.S. Pat. No. 6,202,642 discloses a system for dispensing medication having an electronics module that obtains information related to patient usage of medicine from a medicine canister.

U.S. Pat. No. 4,942,544 discloses a medication clock for producing a record of a patient in complying with a medication schedule. A data base is provided which stores the time and date of each medication that the patient takes including those medications taken in response to an alarm by the clock as well as medications taken by the choice of the patient.

U.S. Pat. No. 5,925,021 discloses a medication delivery device, such as a pen-type injector, jet injector, medication pump, inhaler, spray or the like, that has a microprocessor coupled to the device that records the date, the time, and the amount of each medication administration. The microprocessor may also be coupled to a display to indicate the amount of medication to be administered.

U.S. Pat. Nos. 5,720,733, 5,792,117 and 5,782,814 disclose an apparatus for electrically determining and recording the dose of an agent delivered with a syringe of the type having a barrel for holding the agent and a plunger for expelling the agent. The calculated dose may be recorded by a digital memory unit.

U.S. Pat. No. 5,020,527 discloses a device for inhaling medicine from an inhalation canister with a spray stem. It includes a counter and a timer. The counter is connected to the main body for displaying a count of total activations of an inhalation canister and is advanced by each activation of an inhalation canister. The timer keeps track of time between inhalations for the user.

The above discussed patents are incorporated herein by reference. Despite the various existing systems there is still a need for a simple efficient patient and medical therapy compliance management system.

### SUMMARY OF THE INVENTION

The problems set out above are solved by the present invention. A medical therapy compliance management system according to the present invention manages a patient's use of a medical therapy device, such as a nebulizer, in which treatments are to be provided at predetermined intervals. The system may be used alone or essentially with any medical device, and even combinations thereof. The medical therapy management system may include an interactive voice response system and a compliance controller. The interactive voice response system obtains data directly from one or more patients, wherein the interactive voice response system will contact each patient based upon medication related parameters set by the caregiver. Medication related parameters refer to all parameters associated with the actual medication taken by a patient, medical (or physical) therapy applied to a patient, or the medical equipment for applying the medical or physical therapy to a patient. The medication related parameters may include contacting the patient at least a predetermined period from the last confirmed refill of medication, a predetermined period from the last interactive voice response approving or disapproving a refill, and a predetermined period following no response to an interactive voice response refill request, wherein the predetermined periods are set by the caregiver. The compliance controller is coupled to the interactive voice response system, receives data from the interactive voice response system, and is configured to generate notifications to a given patient and or caregiver based upon the data received. The compliance controller can maintain and generate reports for the caregiver (or others) based upon the data received from the interactive voice response system.

A compliance monitor may be coupled to the medical therapy device of one or more patients, if needed, to determine when a medical treatment is provided to the patient. The compliance controller of the present invention may also receive data from the compliance monitor, wherein one or more parameters of the medical therapy may be calculated by the data from the compliance monitor and the interactive voice response system, and wherein the compliance controller generates notification to a caregiver when a measured or calculated parameter is outside of a preset threshold. The interactive voice response system may also generate patient notifications based upon data obtained from the patient monitor.

The compliance controller may receive data indicative of a medical treatment and may generate a reminder to the patient of future scheduled treatments. The compliance controller may track the treatments provided and may adjust the reminders accordingly. The medical therapy compliance management system of the present invention may track certain parameters indicative of clinical or transactional events within the healthcare process. If any one of the measured or calculated parameters is outside a predetermined threshold the system may notify a patient and or caregiver.

One aspect of the present invention provides an electrical medical therapy device, a compliance monitor, and a compliance controller. The electrical medical therapy device is for providing a plurality of medical treatments to a patient, in which the treatments are originally scheduled to be provided at predetermined intervals. The electrical medical therapy device may be a nebulizer, or the like. The compliance monitor is coupled to the medical therapy device and configured to determine when a medical treatment is provided by the medical therapy device. The compliance controller is coupled to the compliance monitor and receives data from the compliance monitor indicative of a medical treatment. The compliance controller is configured to generate a reminder to the patient of future scheduled treatments, wherein the compliance controller tracks the treatments provided by the medical therapy device and adjusts the generation of the reminders based upon the treatments provided. For example, the compliance controller may adjust the generation of the reminders based upon the treatments provided by at least omitting a scheduled reminder when a patient takes a prescheduled treatment in advance of a scheduled reminder, and delaying a scheduled reminder when such a reminder is within a preset time of a tracked treatment. As a non-limiting illustration, if the patient is to take a treatment at 2:00 P.M. the caregiver may have the system set to issue a reminder at 2:30 P.M. that will only be issued if the system senses that the patient has not taken the treatment by the time of the reminder. As discussed above, where the compliance monitor is used together with the interactive voice response system, the input from the compliance monitor may prompt an appropriate patient call from the interactive voice response system according to the parameters set by the caregiver.

In a respiratory therapy device, such as a nebulizer, the compliance monitor may include a sensor determining the filling of the nebulizer with liquid medication, a sensor determining the atomization of liquid medication in the nebulizer, a sensor determining patient breathing through a patient breathing circuit of the nebulizer, a sensor determining the status of a motor of the nebulizer, or combinations thereof. For example, a measured parameter may include a measurement of the activation of the motor of the nebulizer, wherein the caregiver sets a minimum duration of a given treatment that is to be considered a valid treatment, and the number of valid treatments is recorded by the compliance controller which calculates a % compliant amount and generates reports for the caregiver. Where the electrical medical therapy device is one of a plurality of distinct respiratory therapy devices or combinations thereof, the compliance monitor may include a sensor determining current draw of the electrical medical therapy device and wherein the compliance controller can distinguish which specific respiratory therapy device or combination thereof is coupled to the compliance monitor.

The medical therapy compliance management system of the present invention may provide a compliance monitor and a compliance controller integral with a nebulizer. The caregiver may set treatment thresholds with the compliance monitor configured to determine when a medical treatment is provided by the nebulizer. The compliance controller is configured to generate a notification to a caregiver when a measured or calculated parameter maintained by the compliance controller and associated with the medical treatments is outside of the predetermined threshold. The parameter maintained by the compliance controller may include at least one of a rolling average of % compliance, a number of consecutive missed treatments, % consumption of a refill amount of medication, and patient responses, wherein the patient responses may be data collected from a patient through an interactive voice response system.

The present invention provides a method of tracking patient compliance with a respiratory treatment device comprising the steps of: setting a minimum duration for a respiratory treatment on the respiratory treatment device, wherein respiratory treatments of a duration longer than the minimum are valid treatments and respiratory treatments on the respiratory device of a duration less than the minimum are non-valid treatments, monitoring duration of activation of the respiratory device, wherein activation periods longer than the minimum duration are recorded as compliant treatments, calculating % compliance based upon the recorded compliant treatments and a predetermined schedule of treatments; and generating reports and or notifications for caregivers or patients. As discussed above, the generation of reports and or notifications for caregivers or patients may include generating a reminder to the patient of scheduled treatments, and may include the step of adjusting the generation of the reminders based upon the treatments provided, and may include generating a notification to a caregiver when a measured parameter associated with the medical treatments is outside of a predetermined threshold.

The electronic treatment monitoring device adapted for attachment to, or as an integral component of, any medical device according to the present invention may include a visual display, audible alarms and tactile pushbuttons. The present invention has the capabilities to determine when treatment has been conducted and record all therapy instances. The invention may transmit the resultant records via telephone through the interactive voice response system or through the caregiver, network, internet, smartcard or through wireless telecommunication technology to a central server device, or the compliance controller, designed to present the resultant information to authorized persons such as a physician, therapist, caregiver or the patient. The device, by receiving scheduled times for patient treatments, may remind the patient when a treatment time has approached and provides timely feedback to the patient and authorized caregivers regarding compliance behavior.

The present system provides the caregiver with a large amount of tools to design, modify and implement a specific medication and compliance management system that is particularly suited for a given set of patients. These and other advantages of the present invention will be clarified in the description of the preferred embodiment taken together with the attached figures wherein like reference numerals represent like elements throughout.

### BRIEF DESCRIPTION OF THE DRAWINGS

FIG. 1 is a schematic view of a medical therapy compliance management system according to the present invention; and

FIG. 2 is a view of an input screen for setting compliance thresholds by the caregiver in the medical therapy compliance management system according to the present invention;

FIG. 3 is a schematic flowchart of one implementation of the medical therapy compliance management system according to the present invention; and

FIGS. 4a-h are schematic illustrations of various compliance monitors of the medical therapy compliance management system according to the present invention.

### DETAILED DESCRIPTION OF THE PREFERRED EMBODIMENT

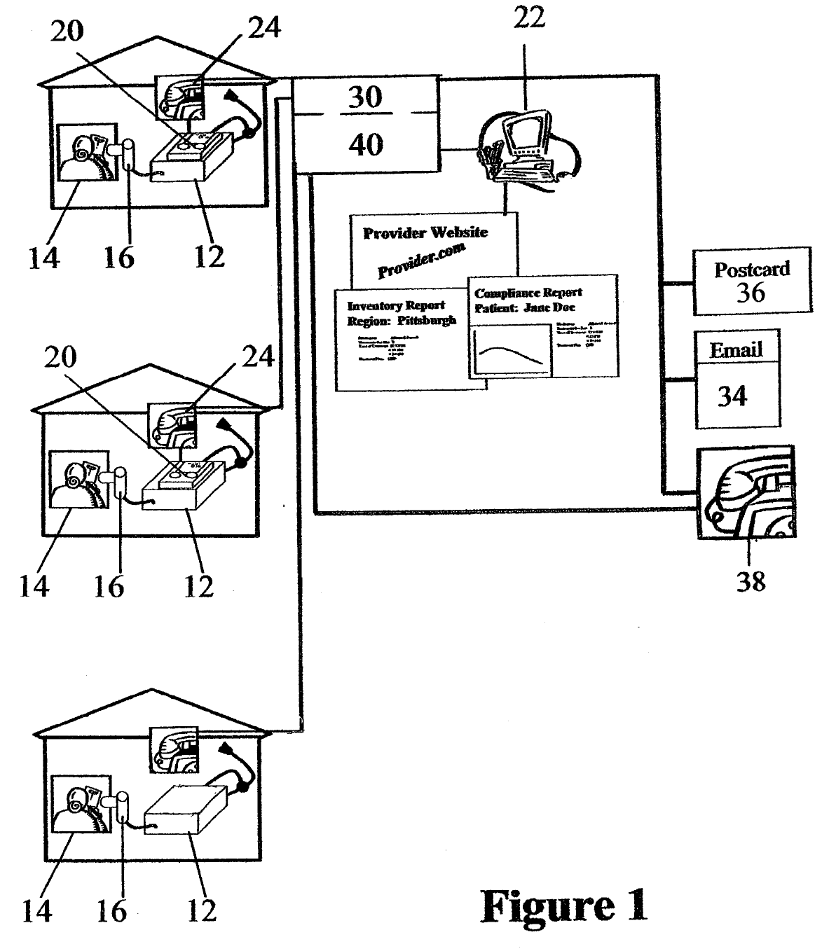

FIG. 1 is a schematic view of a medical therapy compliance management system 10 according to the present invention. The medical therapy compliance management system 10 is utilized with at least one, but preferably a plurality of patients 14 each using an electrical medical therapy device, such as an atomizer or a nebulizer 12, for providing a plurality of medical treatments to a patient 14, in which the treatments are originally scheduled to be provided at predetermined intervals. The medical therapy compliance management system 10 can effectively be used with essentially any medical device, or combinations thereof, or without any associated medical device as will be explained hereinafter. The patient 14 uses the nebulizer 12 to deliver respiratory treatments as prescribed. Respiratory treatments are administered by having the atomizer chamber of the nebulizer 12 filled with medication, turning on the nebulizer 12 and having the patient breath through the breathing circuit 16. The medication will be delivered to the patient 14 as known in the art.

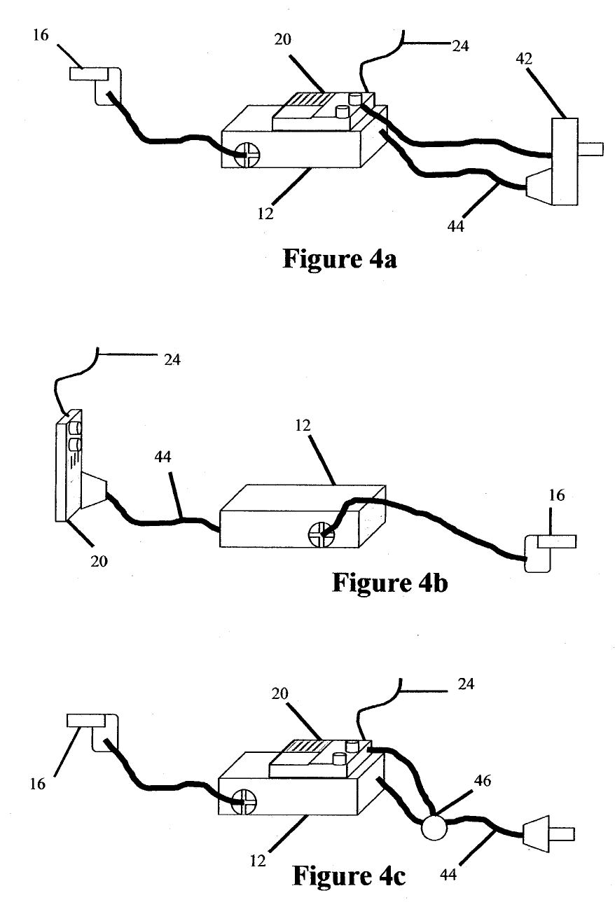

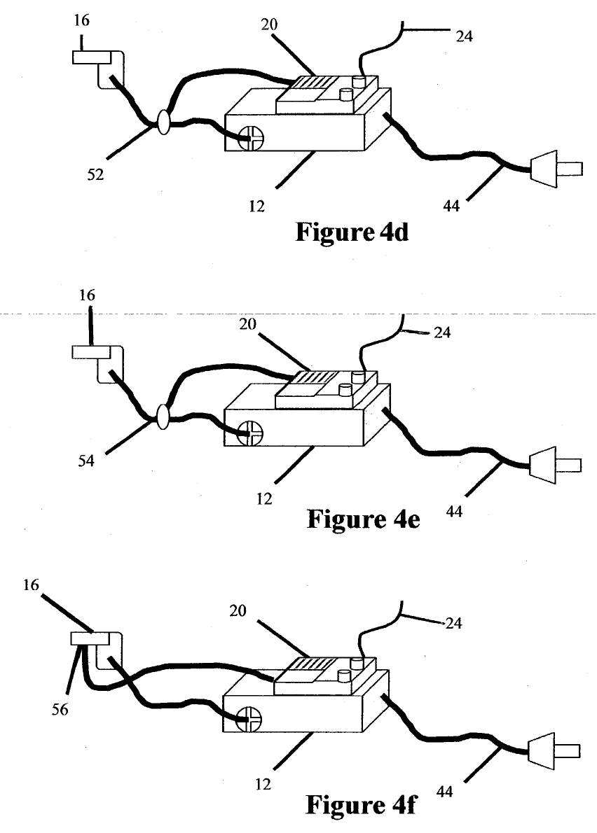

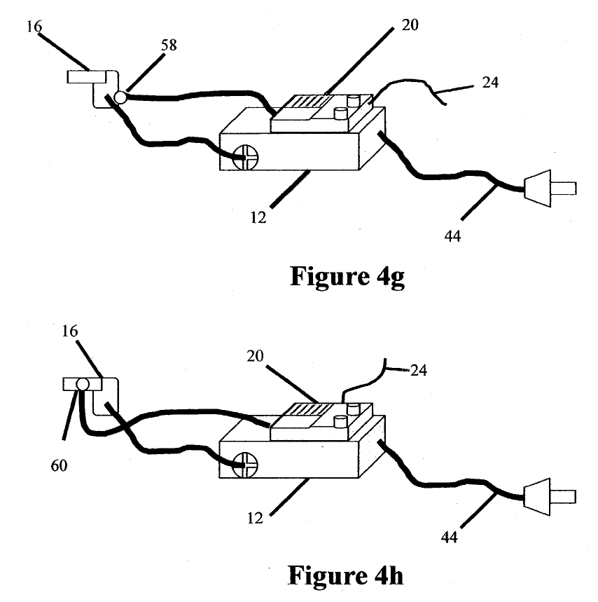

The medical therapy compliance management system 10 may include, as needed for specific patients 14, a compliance monitor 20 coupled to the medical therapy device, e.g. the nebulizer 12 or other respiratory therapy device, and configured to determine when a medical treatment is provided by the medical therapy device. Specifically the compliance monitor 20 will include a sensor sensing use or operation of the medical device, a number of representative examples of such sensors are described below in connection with FIGS. 4a-4h.The compliance monitor 20 may be made integral with the medical therapy device, such as the nebulizer 12. The compliance monitor 20 includes a microprocessor to receive and manage patient treatment or reminder instructions and to send sensed parameters. The compliance monitor 20 may include a LED or other video display for the patient 14 as well as appropriate input and control buttons for the patient 14, the caregiver 22 or others. Additionally the compliance monitor 20 includes a communications link 24, e.g. a telephone line, wireless communication, etc, for communication with the remainder of the medical therapy compliance management system 10.

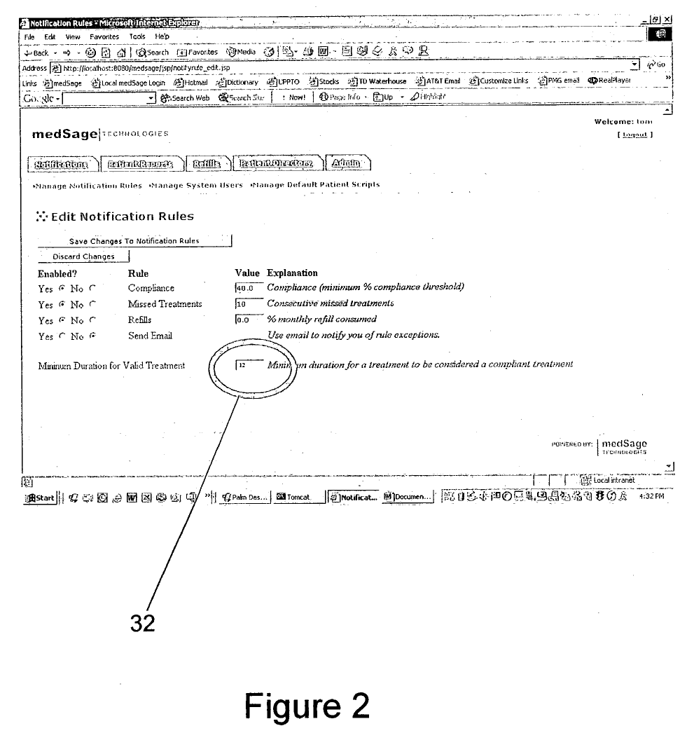

The medical therapy compliance management system 10 includes a compliance controller 30 associated with all of the patients 14 and coupled to each compliance monitor 20 through the associated communications link 24. The compliance controller 30, or central controller, receives data from the compliance monitor 20 indicative of a medical treatment by the medical therapy device. The medical therapy compliance management system 10 is adapted to have patient management rules predetermined by the caregiver 22. FIG. 2 illustrates a representative set of rules or thresholds 32 which may be set by the caregiver 22. The thresholds may be, for the nebulizer 12, a minimum amount of total patient compliance (i.e. what is the ratio of actual treatment taken to prescribed treatments), a number of consecutive missed treatments, and percentage of medication allotment consumed (i.e. how close is the patient to needing a refill). As illustrated in FIG. 2 the medical therapy compliance management system 10 may have the caregiver 22 input the thresholds 32 through a web-based user interface which is connected to the compliance controller 30. Any other user interface may similarly be utilized. The compliance controller 30 generates notification to a caregiver 22 and or the patient 12 when a measured, or calculated parameter, is outside of a preset threshold 32. The notification may be by e-mail 34, postcard 36, telephone 38 or any acceptable notification scheme. The compliance controller 30 can maintain, generate or transmit reports to the caregiver 22 or others for each, or combination of, the patients 14 based upon all of the data received. The specific reports and transmission schedules can be designed and set by the caregiver 22.

The compliance controller 30 may also generate reminders of upcoming treatments to the patient 12, with these reminders being sent in the same manner as the notifications discussed above. The compliance controller 30 will also adjust the generation of the reminders based upon the treatments provided, as sensed by the compliance monitor 24. The compliance controller 30 may adjust the generation of the reminders, for example, by at least omitting a scheduled reminder when a patient takes a prescheduled treatment in advance of a scheduled-reminder, and delaying a scheduled reminder when such a reminder is within a preset time of a tracked treatment. The caregiver 22 may set the “preset time” amounts in the same manner as setting the thresholds 32 above. It is anticipated that many of the preset times and thresholds will be standard for a specific treatment regimen. These standard presets and thresholds may thereby be applied as a set to a patient, or they may be set individually as needed.

The medical therapy compliance management system 10 according to the present invention provides that the caregiver 22 may set a threshold 32 associated with a minimum duration of a given treatment that is to be considered a valid or compliant treatment. For example, any treatment lasting over 12 minutes may be considered as a valid treatment and those lasting under 12 minutes may ignored (i.e. no treatment). The specific amounts, of course, will vary between applications. Further additional logic in the compliance controller 30 may account for very short gaps in treatment sessions. As discussed above, the number of valid treatments is recorded by the compliance controller 30 which calculates a % compliant amount and generates reports for the caregiver 22 and/or the patient 14.

The medical therapy compliance management system 10 as described provides a method of tracking patient compliance with a respiratory treatment device as follows. A minimum duration for a respiratory treatment on the respiratory treatment device is set, wherein respiratory treatments of a duration longer than the minimum are valid treatments and respiratory treatments on the respiratory device of a duration less than the minimum are non-valid treatments. The duration of activation of the respiratory device is monitored by the compliance monitor 20, wherein activation periods longer than the minimum duration are recorded as valid treatments by the compliance controller 30. The % compliance is calculated by the compliance controller 30 based upon the recorded compliant or valid treatments and a predetermined schedule of treatments. The medical therapy compliance management system 10 generates reports and or notifications for caregivers 22 or patients 14, accordingly. The generation of reports and or notifications for caregivers 22 or patients 14 can include generating a reminder to the patient 14 of scheduled treatments, and further include adjusting the generation of the reminders based upon the treatments provided. The generation of reports and or notifications for caregivers 22 or patients 14 may include generating a notification to a caregiver 22 when a measured parameter associated with the medical treatments is outside of a predetermined threshold 32. Further the calculated or measured parameters may include a rolling average of % compliance, a number of consecutive missed treatments, % consumption of a refill amount of medication, and patient responses.

Another critical aspect or feature of the medical therapy compliance management system 10 is the use of interactive voice response technology in or with the compliance controller 30. The compliance controller 30 may include an interactive voice response system 40 that can be utilized to automate the contacting and obtaining of certain information or data directly from the patient 14 through the telephone 38. The interactive voice response system 40 will contact the patients 14 according to preset medication related parameters set by the caregiver 22. These parameters may be, for example, to contact the patient 14 at least a predetermined period from the last confirmed refill of medication, a predetermined period from the last interactive voice response approving or disapproving a refill, and a predetermined period following no response to an interactive voice response refill request. The predetermined periods are set by the caregiver 22 in the manner discussed above.

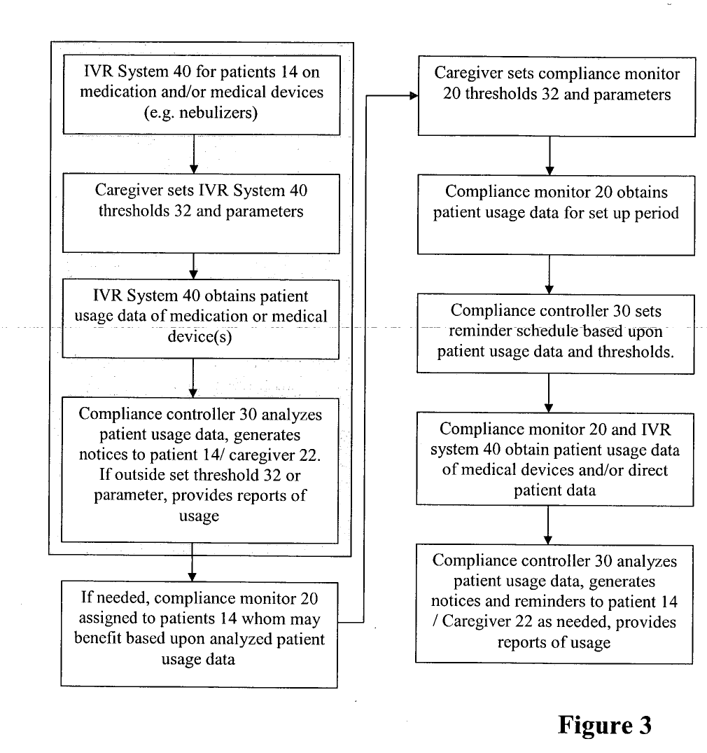

As shown in FIG. 1, not all of the patients 14 require the compliance monitor 20 on the associated medical device. It is anticipated that for many specific patients 14 the interactive voice response system 40 and the compliance controller 30 alone (i.e. without a compliance monitor 20 or associated link 24) will be sufficient for medication and compliance management. As shown in FIG. 1 and FIG. 3, the controller 30 and interactive voice response system 40 alone can operate as a medication and compliance management system for patients. In such uses, the system is not limited to medical devices but may be used to manage patient care with medication or therapy that is not associated with use of an electronic medical device. The use of the interactive voice response system 40 and controller 30 alone will greatly improve patient management. For example, the compliance controller 30 with the interactive voice response system 40 may be implemented with a series of patients 14 each utilizing a medical device, such as a nebulizer 12, without the associated compliance monitors 20. There would, of course be no input to the compliance controller 30 regarding the actual usage. The data obtained by the interactive voice response system 40 will be used to manage medication and patient compliance. The data obtained by the interactive voice response system 40 can be used to generate notifications to the patient 14 or caregiver 22 as set by the caregiver 22. The interactive voice response system 40 may be used for certain patient 14 or caregiver 22 notifications (e.g. “patient prescription change”, “patient condition change”, “patient refill refusal”, “patient unable to be reached”). The data obtained by the interactive voice response system 40 can be utilized by the compliance controller 30 to maintain, generate, and transmit reports to the caregiver 22. As discussed above, the interactive voice response system 40 will contact the patients 14 or caregiver 22 for data acquisition or notification according to preset medication related parameters set by the caregiver 22. Consequently, the uses of the interactive voice response system 40 will be set by the caregiver 22.

The data obtained by the interactive voice response system 40 may be used to determine which patients 12 should receive a more comprehensive compliance system that incorporates the compliance monitor 20. In other words, if the refills are not being ordered on a regular basis, or other data received from the patient 14 through the interactive voice response system 40 is not acceptable (e.g. the patient indicates that his or her condition is not improving as expected over a period of time), then a compliance monitor 20 can be attached to the medical device for that patient 14 (or a device incorporating the monitor 20 can be sent to the patient 14).

Additionally, the medical therapy compliance management system 10 may be used by first deploying the compliance monitor 20 with no reminders to the patient for a set up period, such as a week. The compliance monitor 20 can then be utilized to collect the patient behavioral data (i.e. how does the patient 14 typically utilize the medical device). Following the set up period, a reminder schedule can be implemented by the medical therapy compliance management system 10 based upon the observed usage characteristics of the specific patient 14.

FIGS. 4a-h are schematic illustrations of various compliance monitors 20 of the medical therapy compliance management system 10 according to the present invention. FIG. 4a illustrates a compliance monitor 20 with a current sensor 42 associated with the power cord 44 of the medical device, such as the nebulizer 12. The current sensor 42 will sense the current draw when the nebulizer 12 is in operation. FIG. 4b illustrates a compliance monitor 20 with a current sensor 42 built directly into the compliance monitor 20. FIG. 4c illustrates a compliance monitor 20 with a magnetic field sensor 46 (e.g. an induction coil) associated with the power cord 44 of the medical device, such as the nebulizer 12. FIGS. 4d-f illustrate compliance monitors 20 with various pressure sensors 52, 54 and 56 associated with the breathing circuit 16 of the nebulizer 12. Sensor 52 utilizes a “T” fitting, sensor 54 utilizes a tubing cuff and sensor 56 is a pressure sensor in the atomizer of the device. FIGS. 4g and h illustrate compliance monitors 20 with fluid sensors 58 and 60, sensing, respectively, the fluid level of medication and the presence of aerosolized medication. All of the sensors illustrated in FIGS. 4a-h are merely representative examples of what may be utilized to determine if the device is turned on. Other sensors are possible.

An advantage with utilizing the current sensors shown in FIGS. 4a-c is that the medical therapy compliance management system 10 can be used to differentiate and identify what specific medical device is being utilized. It is anticipated that the compliance monitor 20 will be used with one of one of a plurality of distinct respiratory therapy devices and combinations thereof having distinct identifiable current draws. With the compliance monitor 20 including a sensor determining current draw of the electrical medical therapy device, the compliance controller 30 can distinguish which specific respiratory therapy device or combination thereof is coupled to the compliance monitor 20. FIG. 3 is a schematic flowchart of one implementation of the medical therapy compliance management system 10 according to the present invention.

The other advantage of the present invention will be apparent to those in the art. The object of the present invention is to give the caregiver 22 very broad tools in the system 10 for managing medication and patient compliance. The invention has been described with reference to the preferred embodiment, but is not intended to be limited thereby. Obvious modifications and alterations will occur to others upon reading and understanding the proceeding detailed description. It is intended that the invention be construed as including all such modifications and alterations. The scope of the present invention is defined by the appended claims and equivalents thereto.

What is claimed is:

1. A medical therapy compliance management system comprising:
An electrical medical therapy device for providing a plurality of medical treatments to a patient, in which the treatments are originally scheduled to be provided at predetermined intervals;
a compliance monitor coupled the one medical therapy device and configured to determine when a medical treatment is provided by the medical therapy device;
a compliance controller coupled to the compliance monitor and receiving data from the compliance monitor indicative of a medical treatment, the compliance controller configured to generate a reminder to the patient of future scheduled treatments, wherein the compliance controller tracks the treatments provided by the medical therapy device and adjusts the generation of the reminders based upon the treatments provided.

2. The medical therapy compliance management system of claim 1 wherein the electrical medical therapy device is a nebulizer providing respiratory treatment to the patient.

3. The medical therapy compliance management system of claim 2 wherein the compliance monitor includes one of the group comprising a sensor determining the filling of the nebulizer with liquid medication; a sensor determining the atomization of liquid medication in the nebulizer; a sensor determining patient breathing through a patient breathing circuit of the nebulizer; a sensor determining the status of a motor of the nebulizer; and combinations thereof.

4. The medical therapy compliance management system of claim 2 wherein the compliance controller adjusts the generation of the reminders based upon the treatments provided by at least
omitting a scheduled reminder when a patient takes a prescheduled treatment in advance of a scheduled reminder, and
delaying a scheduled reminder when such a reminder is within a preset time of a tracked treatment.

5. The medical therapy compliance management system of claim 2 wherein the data from the compliance monitor is one measured parameter tracked by the compliance controller, wherein one or more parameters of the medical therapy may be calculated by the data from the compliance monitor, and wherein the compliance controller generates notification to a caregiver when a measured or calculated parameter is outside of a preset threshold.

6. The medical therapy compliance management system of claim 5 wherein the measured parameters include a measurement of the activation of the motor of the nebulizer, wherein the caregiver sets a minimum duration of a given treatment that is to be considered a valid treatment, and the number of valid treatments is recorded by the compliance controller which calculates a % compliant amount and generates reports for the caregiver.

7. The medical therapy compliance management system of claim 5 wherein the measured parameters include data collected from a patient through an interactive voice response system, wherein the interactive voice response system contacts patients based upon medication related parameters set by the caregiver.

8. The medical therapy compliance management system of claim 7 wherein the interactive voice response system will contact the patient at least:
a predetermined period from the last confirmed refill of medication,
a predetermined period from the last interactive voice response approving or disapproving a refill, and
a predetermined period following no response to an interactive voice response refill request,
wherein the predetermined periods are set by the caregiver.

9. The medical therapy compliance management system of claim 2 wherein the compliance monitor includes a sensor determining current draw of the nebulizer.

10. The medical therapy compliance management system of claim 2 wherein the compliance monitor and the compliance controller are integral with the nebulizer.

11. The medical therapy compliance management system of claim 1 wherein the electrical medical therapy device is one of a plurality of distinct respiratory therapy devices and combinations thereof, and wherein the compliance monitor includes a sensor determining current draw of the electrical medical therapy device and wherein the compliance controller can distinguish which specific respiratory therapy device or combination thereof is coupled to the compliance monitor.

12. A medical therapy compliance management system comprising:
a nebulizer for providing a plurality of respiratory medical treatments to a patient scheduled to be provided at predetermined intervals, wherein a caregiver sets a minimum duration of a given treatment that is to be considered a valid medical treatment;
a compliance monitor coupled to the nebulizer and configured to determine when a medical treatment is provided by the nebulizer;
a compliance controller coupled to the compliance monitor and receiving data from the compliance monitor indicative of a medical treatment, the compliance controller tracking compliant medical treatments and configured to generate a notification to a caregiver when a measured or calculated parameter maintained by the compliance controller and associated with the medical treatments is outside of a predetermined threshold.

13. The medical therapy compliance management system of claim 12 wherein the parameter maintained by the compliance controller includes at least one of
a rolling average of % compliance,
a number of consecutive missed treatments,
% consumption of a refill amount of medication, and
patient responses.

14. The medical therapy compliance management system of claim 13 wherein the measured parameters include patient responses, and wherein the patient responses are data collected from a patient through an interactive voice response system, and wherein the notifications include-at least one of
notification of a patient prescription change,
notification of patient condition change,
notification of patient refill refusal,
notification of inability to reach patient.

15. The medical therapy compliance management system of claim 14 wherein the interactive voice response system will contact the patient based upon predetermined medication related parameters including contacting the patient at least:
a predetermined period from the last confirmed refill of medication,
a predetermined period from the last interactive voice response approving or disapproving a refill, and
a predetermined period following no response to an interactive voice response refill request,
wherein the predetermined periods are set by the caregiver.

16. The medical therapy compliance management system of claim 12 wherein the compliance controller is configured to generate a reminder to the patient of scheduled treatments and wherein the compliance controller adjusts the generation of the reminders based upon the compliant treatments recorded.

17. The medical therapy compliance management system of claim 12 wherein the compliance monitor includes a sensor determining current draw of the nebulizer.

18. The medical therapy compliance management system of claim 12 wherein the compliance monitor and the compliance controller is integral with the nebulizer.

19. A method of tracking patient compliance with a respiratory treatment device comprising the steps of:
setting a minimum duration for a respiratory treatment on the respiratory treatment device, wherein respiratory treatments of a duration longer than the minimum are valid treatments and respiratory treatments on the respiratory device of a duration less than the minimum are non-valid treatments;
monitoring duration of activation of the respiratory device, wherein activation periods longer than the minimum duration are recorded as compliant treatments;
calculating % compliance based upon the recorded compliant treatments and a predetermined schedule of treatments; and
generating reports and or notifications for caregivers or patients.

20. The method of tracking patient compliance according to claim 19 wherein the generation of reports and or notifications for caregivers or patients includes generating a reminder to the patient of scheduled treatments, and further includes the step of adjusting the generation of the reminders based upon the treatments provided.

21. The method of tracking patient compliance according to claim 19 wherein the generation of reports and or notifications for caregivers or patients includes generating a notification to a caregiver when a measured parameter associated with the medical treatments is outside of a predetermined threshold.

22. The method of tracking patient compliance according to claim 21 wherein the measured parameters include
a rolling average of % compliance,
a number of consecutive missed treatments,
% consumption of a refill amount of medication, and
patient responses.

23. The method of tracking patient compliance according to claim 22 wherein the patient response are obtained through an interactive voice response system.

24. The method of tracking patient compliance according to claim 19 wherein the monitoring is through a measuring of the current draw of the respiratory treatment device.

25. The method of tracking patient compliance according to of claim 24 further including the step of distinguishing between a number of distinct respiratory devices utilized based upon the monitored current draw.

26. A medical therapy compliance management system comprising:
an electrical medical therapy device for providing a plurality of medical treatments to a patient, in which the treatments are originally scheduled to be provided at predetermined intervals;
a compliance monitor coupled to the medical therapy device and configured to determine when a medical treatment is provided by the medical therapy device;
an interactive voice response system for obtaining data from the patient; and
a compliance controller coupled to the compliance monitor and the interactive voice response system, the compliance controller receiving data from the compliance monitor indicative of a medical treatment and receiving data from the interactive voice response system, the compliance controller configured to generate notifications to the patient and or caregiver based upon the data received.

27. The medical therapy compliance management system of claim 26 wherein the interactive voice response system will contact the patient at least:
a predetermined period from the last confirmed refill of medication,
a predetermined period from the last interactive voice response approving or disapproving a refill, and
a predetermined period following no response to an interactive voice response refill request,
wherein the predetermined periods are set by the caregiver.

28. The medical therapy compliance management system of claim 26 wherein the compliance controller generates a reminder to the patient of scheduled treatments, wherein the compliance controller adjusts the generation of the reminders based upon the treatments provided, and wherein the compliance controller generates a notification to a caregiver when a measured parameter maintained by the compliance controller and associated with the medical treatments is outside of a predetermined threshold.

29. The medical therapy compliance management system of claim 26 wherein the medical therapy device is a nebulizer and wherein the compliance monitor includes a sensor determining current draw of the nebulizer.

30. A nebulizer monitoring device for a nebulizer comprising:
a compliance monitor coupled to the nebulizer and configured to determine when a medical treatment is provided by the nebulizer;
a compliance controller coupled to the compliance monitor and receiving data from the compliance monitor indicative of a medical treatment, the compliance controller tracking compliant medical treatments and configured to generate one of
a notification to a caregiver when a measured parameter maintained by the compliance controller and associated with the medical treatments is outside of a predetermined threshold, and
a reminder to the patient of scheduled treatments while adjusting the generation of the reminders based upon the treatments recorded.

31. The nebulizer monitoring device of claim 30 wherein the monitoring device is integral with the nebulizer.

32. The nebulizer monitoring device of claim 30 wherein the compliance monitor includes a sensor determining current draw of the nebulizer, and wherein the monitoring device is adapted to be coupled to additional respiratory devices wherein the compliance controller distinguishes between the distinct respiratory devices utilized based upon the monitored current draw.

33. A medical therapy management system for managing medical therapy to a patient, the system comprising:
an interactive voice response system for obtaining data from the patient, wherein the interactive voice response system will contact the patient based upon medication related parameters set by the caregiver; and
a compliance controller coupled to the interactive voice response system, the compliance controller receiving data from the interactive voice response system, the compliance controller configured to generate notifications and or reports to the patient and or caregiver based upon the data received.

34. The medical therapy management system of claim 33 wherein the interactive voice response system monitors the patient for prescription changes, patient health condition, and medication refill requirements.

35. The medical therapy management system of claim 33 wherein the interactive voice response system has parameters that prompt contacting of the patient at least a predetermined period from the last confirmed refill of medication, a predetermined period from the last interactive voice response approving or disapproving a refill, and a predetermined period following no response to an interactive voice response refill request, wherein the predetermined periods are set by the caregiver 36. The medical therapy management system of claim 33 wherein the interactive voice response system can generate patient and or caregiver notifications based upon data received from a compliance monitor associated with a medical device.
